(19) United States
(12) Patent Application Publication
(10) Pub. No.: US 2002/0173491 A1
(43) Pub. Date: Nov. 21, 2002

(54) DAPD combination therapy with inosine monophosphate dehydrogenase inhibitor

(76) Inventors: Philip A. Furman, Durham, NC (US); Katyna Borroto-Esoda, Raleigh, NC (US)

Correspondence Address: KING & SPALDING, 191 PEACHTREE STREET, N.E., ATLANTA, GA, 30303-1763, US

(21) Appl. No.: 10/023,636

(22) Filed: Dec. 17, 2001

Related U.S. Application Data
(60) Provisional application No. 60/256,068, filed on Dec. 15, 2000.
(60) Provisional application No. 60/272,605, filed on Mar. 1, 2001.

(51) Int. Cl. A61K 31/70; A61K 31/675; A61K 31/52; A61K 31/353

(52) U.S. Cl. 514/81; 514/263.23; 514/43; 514/469

(57) ABSTRACT

It has been unexpectedly found that a drug resistant strain of HIV exhibits the behavior of drug-naïve virus when given the combination of a β-D-1,3-dioxolanyl nucleoside and an IMPDH inhibitor. In one nonlimiting embodiment, the HIV strain is resistant to a β-D-1,3-dioxolanyl nucleoside.

### FIELD OF THE INVENTION

The present invention relates to pharmaceutical compositions and methods for the treatment or prophylaxis of human immunodeficiency virus (HIV) infection in a host comprising administering such compositions. This application claims priority to U.S. provisional application No. 60/256,068 filed on Dec. 15, 2000 and to U.S. provisional application No. 60/272,605 filed on Mar. 1, 2001.

### BACKGROUND OF THE INVENTION

AIDS, Acquired Immune Deficiency Syndrome, is a catastrophic disease that has reached global proportions. From July 1998 through June 1999 a total of 47,083 AIDS cases were reported in the US alone. With more than 2.2 million deaths in 1998, HIV/AIDS has now become the fourth leading cause of mortality and its impact is going to increase. The death toll due to AIDS has reached a record 2.6 million per year, while new HIV infections continued to spread at a growing rate, according to a recent UNAIDS report.

AIDS was first brought to the attention of the Center for Disease Control and Prevention (CDC) in 1981 when seemingly healthy homosexual men came down with Karposi's Sarcoma (KS) and Pneumocystis Carinii Pneumonia (PCP), two opportunistic diseases that were only known to inflict immuno-deficient patients. A couple of years later, the causitive agent of AIDS, a lymphoadenopathy associated retrovirus, the human immunodefieciency virus (HIV) was isolated by the Pasteur Institute in Paris, and later confirmed by an independent source in the National Cancer Institute of the United States.

In 1986, at the second International Conference on AIDS in Paris, preliminary reports on the use of a drug against AIDS were presented. This drug, 3′-azido-3′-deoxy-thymidine (AZT, Zidovudine, Retrovir), was approved by the Food And Drug Administration (FDA) and it became the first drug to be used in the fight against AIDS. Since the advent of AZT, several nucleoside analogs have been shown to have potent antiviral activity against the human immunodeficiency virus type I (HIV-I). In particular, a number of 2′,3′-dideoxy-2′,3′-didehydro-nucleosides have been shown to have potent anti-HIV-1 activity. 2′,3′-Dideoxy-2′,3′-didehydro-thymidine (“D4T”; also referred to as 1-(2,3-dideoxy-β-D-glycero-pent-2-eno-furanosyl)thymine)) is currently sold for the treatment of HIV under the name Stavudine by Bristol Myers Squibb.

It has been recognized that drug-resistant variants of HIV can emerge after prolonged treatment with an antiviral agent. Drug resistance most typically occurs by mutation of a gene that encodes for an enzyme used in viral replication, and most typically in the case of HIV, reverse transcriptase, protease or DNA polymerase. Recently, it has been demonstrated that the efficacy of a drug against HIV infection can be prolonged, augmented, or restored by administering the compound in combination or alternation with a second, and perhaps third, antiviral compound that induces a different mutation from that caused by the principle drug. Alternatively, the pharmacokinetics, biodistribution or other parameter of the drug can be altered by such combination or alternation therapy. In general, combination therapy is typically preferred over alternation therapy because it induces multiple simultaneous pressures on the virus. One cannot predict, however, what mutations will be induced in the HIV-1 genome by a given drug, whether the mutation is permanent or transient, or how an infected cell with a mutated HIV-1 sequence will respond to therapy with other agents in combination or alternation. This is exacerbated by the fact that there is a paucity of data on the kinetics of drug resistance in long-term cell cultures treated with modern antiretroviral agents.

HIV-1 variants resistant to 3′-azido-3′-deoxythymidine (AZT), 2′,3′-dideoxyinosine (DDI) or 2′,3′-dideoxycytidine (DDC) have been isolated from patients receiving long term monotherapy with these drugs (Larder B A, Darby G, Richman D D. Science 1989;243:1731-4; St Clair M H, Martin J L, Tudor W G, et al. Science 1991;253:1557-9; St Clair M H, Martin J L, Tudor W G, et al. Science 1991;253:1557-9; and Fitzgibbon J E, Howell R M, Haberzettl C A, Sperber S J, Gocke D J, Dubin D T. Antimicrob Agents Chemother 1992;36:153-7). Mounting clinical evidence indicates that AZT resistance is a predictor of poor clinical outcome in both children and adults (Mayers D L. Lecture at the Thirty-second Interscience Conference on Antimicrobial Agents and Chemotherapy. (Anaheim, Calif. 1992); Tudor-Williams G, St Clair M H, McKinney R E, et al. Lancet 1992;339:15-9; Ogino M T, Dankner W M, Spector S A. J Pediatr 1993;123:1-8; Crumpacker CS, D′Aquila R T, Johnson V A, et al. Third Workshop on Viral Resistance. (Gaithersburg, Md. 1993); and Mayers D, and the RV43 Study Group. Third Workshop on Viral Resistance. (Gaithersburg, Md. 1993)).

The rapid development of HIV-1 resistance to nonnucleoside reverse transcriptase inhibitors (NNRTIs) has also been reported both in cell culture and in human clinical trials (Nunberg J H, Schleif W A, Boots E J, et al. J Virol 1991;65(9):4887-92; Richman D, Shih C K, Lowy I, et al. Proc Natl Acad Sci (USA) 1991;88:11241-5; Mellors J W, Dutschman G E, Im G J, Tramontano E, Winkler S R, Cheng Y C. Mol Pharm 1992;41:446-51; Richman DD and the ACTG 164/168 Study Team. Second International HIV-1 Drug Resistance Workshop. (Noordwijk, the Netherlands. 1993); and Saag M S, Emini E A, Laskin O L, et al. N Engl J Med 1993;329:1065-1072). In the case of the NNRTI L'697,661, drug-resistant HIV-1 emerged within 2-6 weeks of initiating therapy in association with the return of viremia to pretreatment levels (Saag M S, Emini E A, Laskin O L, et al. N Engl J Med 1993;329:1065-1072). Breakthrough viremia associated with the appearance of drug-resistant strains has also been noted with other classes of HIV-1 inhibitors, including protease inhibitors (Jacobsen H, Craig C J, Duncan I B, Haenggi M, Yasargil K, Mous J. Third Workshop on Viral Resistance. (Gaithersburg, Md. 1993)). This experience has led to the realization that the potential for HIV-1 drug resistance must be assessed early on in the preclinical evaluation of all new therapies for HIV-1.

1,3-Dioxolanyl Nucleosides

The success of various synthetic nucleosides in inhibiting the replication of HIV in vivo or in vitro has led a number of researchers to design and test nucleosides that substitute a heteroatom for the carbon atom at the 3′-position of the nucleoside. Norbeck, et al., disclosed that (+/−)-1-[(2-β, 4-β)-2-(hydroxymethyl)-4-dioxolanyl]thymine (referred to as (+/−)-dioxolane-T) exhibits a modest activity against HIV (EC₅₀ of 20 μM in ATH8 cells), and is not toxic to uninfected control cells at a concentration of 200 μM. Tetrahedron Letters 30 (46), 6246, (1989).

On Apr. 11, 1988, Bernard Belleau, Dilip Dixit, and Nghe Nguyen-Ba at BioChem Pharma filed patent application U.S. Ser. No. 07/179,615 which disclosed a generic group of racemic 2-substituted-4-substituted-1,3-dioxolane nucleosides for the treatment of HIV. The '615 patent application matured into European Patent Publication No. 0 337 713; U.S. Pat. No. 5,041,449; and U.S. Pat. No. 5,270,315 assigned to BioChem Pharma, Inc.

On Dec. 5, 1990, Chung K. Chu and Raymond F. Schinazi filed U.S. Ser. No. 07/622,762, which disclosed an asymmetric process for the preparation of enantiomerically enriched B-D-1,3-dioxolane nucleosides via stereospecific synthesis, and certain nucleosides prepared thereby, including (−)-(2R,4R)-9-[(2-hydroxymethyl)-1,3-dioloan-4-yl]guanine (DXG), and its use to treat HIV. This patent application issued as U.S. Pat. No. 5,179,104.

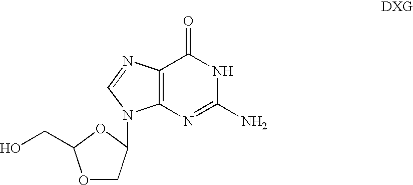

On May 21, 1991, Tarek Mansour, et al., at BioChem Pharma filed U.S. Ser. No. 07/703,379 directed to a method to obtain the enantiomers of 1,3-dioxolane nucleosides using a stereoselective synthesis that includes condensing a 1,3-dioxolane intermediate covalently bound to a chiral auxiliary with a silyl Lewis acid. The corresponding application was filed in Europe as EP 0 515 156.

On Aug. 25, 1992, Chung K. Chu and Raymond F. Schinazi filed U.S. Ser. No. 07/935,515, disclosing certain enantiomerically enriched β-D-dioxolanyl purine compounds for the treatment of humans infected with HIV of the formula:

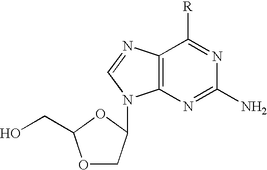

wherein R is OH, Cl, NH₂ or H, or a pharmaceutically acceptable salt or derivative of the compounds optionally in a pharmaceutically acceptable carrier or diluent. The compound wherein R is chloro is referred to as (−)-(2R,4R)-2-amino-6-chloro-9-[(2-hydroxymethyl)-1,3-dioxolan-4-yl]purine. The compound wherein R is hydroxy is (−)-(2R,4R)-9-[(2-hydroxy-methyl)-1,3-dioxolan-4-yl]guanine. The compound wherein R is amino is (−)-(2R,4R)-2-amino-9-[(2-hydroxymethyl)-1,3-dioxolan-4-yl]adenine. The compound wherein R is hydrogen is (−)-(2R,4R)-2-amino-9-[(2-hydroxymethyl)-1,3-dioxolan-4yl]purine. This application issued as U.S. Pat. Nos. 5,925,643 and 5,767,122.

In 1992, Kim et al., published an article teaching how to obtain (−)-L-β-dioxolane-C and (+)-L-β-dioxolane-T from 1,6-anhydro-L-β-glucopyranose. Kim et al., Potent anti-HIV and anti-HBV Activities of (−)-L-β-Dioxolane-C and (+)-L-β-Dioxolane-T and Their Asymmetric Syntheses, Tetrahedron Letters Vol 32(46), pp 5899-6902.

On October 28, 1992, Raymond Schinazi filed U.S. Ser. No. 07/967,460 directed to the use of the compounds disclosed in U.S. Ser. No. 07/935,515 for the treatment of hepatitis B. This application has issued as U.S. Pat. Nos. 5,444,063; 5,684,010; 5,834,474; and 5,830,898.

In 1993, Siddiqui, et al., at BioChem and Glaxo published that cis-2,6-diaminopurine dioxolane can be deaminated selectively using adenosine deaminase. Siddiqui, et al., Antiviral Optically Pure dioxolane Purine Nucleoside Analogues, Bioorganic & Medicinal Chemistry Letters, Vol. 3 (8), pp 1543-1546 (1993).

(−)-(2R,4R)-2-amino-9-[(2-hydroxymethyl)-1,3-dioxolan-4-yl]adenine (DAPD) is a selective inhibitor of HIV-1 replication in vitro as a reverse transcriptase inhibitor (RTI). DAPD is thought to be deaminated in vivo by adenosine deaminase, a ubiquitous enzyme, to yield (−)-β-D-dioxolane guanine (DXG), which is subsequently converted to the corresponding 5′-triphosphate (DXG-TP). Biochemical analysis has demonstrated that DXG-TP is a potent inhibitor of the HIV reverse transcriptase (HIV-RT) with a Ki of 0.019 μM.

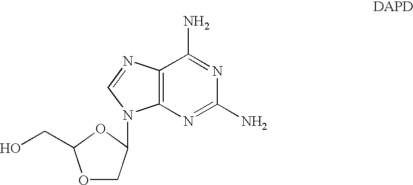

Triangle Pharmaceuticals, Inc. (Durham, N.C.) is currently developing this compound for the treatment of HIV and HBV under license agreement from Emory University in collaboration with Abbott Laboratories, Inc.

Ribavirin

Ribavirin (1-β-D-ribofuranosyl-1,2,4-triazole-3-carboxamide) is a synthetic, non-interferon-inducing, broad spectrum antiviral nucleoside analog sold under the trade name Virazole (The Merck Index, 11th edition, Editor: Budavari, S., Merck & Co., Inc., Rahway, N.J., p1304, 1989). U.S. Pat. No. 3,798,209 and RE29,835 disclose and claim ribavirin. In the United States, ribavirin was first approved as an aerosol form for the treatment of a certain type of respiratory virus infection in children. Ribavirin is structurally similar to guanosine, and has in vitro activity against several DNA and RNA viruses including Flaviviridae (Gary L. Davis Gastroenterology 118:S114-S114, 2000). Ribavirin reduces serum amnino transferase levels to normal in 40% of patients, but it does not lower serum levels of HCV-RNA (Gary L. Davis Gastroenterology 118:S104-S114, 2000). Thus, ribavirin alone is not effective in reducing viral RNA levels. It is being studied in combination with DDI as an anti-HIV treatment. More recently, it has been shown to exhibit activity against hepatitis A, B and C. Since the beginning of the AIDS crisis, people have used ribavirin as an anti-HIV treatment, however, when used as a monotherapy, several controlled studies have shown that ribavirin is not effective against HIV. It has no effect on T4 cells, T8 cells or p24 antigen.

The combination of IFN and ribavirin for the treatment of HCV infection has been reported to be effective in the treatment of IFN naïve patients (Battaglia, A. M. et al., Ann. Pharmacother. 34:487-494, 2000). Results are promising for this combination treatment both before hepatitis develops or when histological disease is present (Berenguer, M. et al. Antivir. Ther. 3(Suppl. 3):125-136, 1998). Side effects of combination therapy include hemolysis, flulike symptoms, anemia, and fatigue (Gary L. Davis. Gastroenterology 118:S104-S114, 2000).

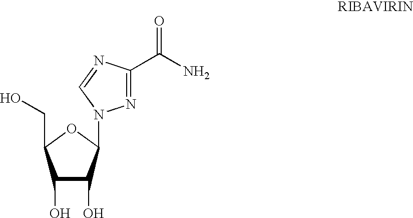

Mycophenolic Acid

Mycophenolic acid (6-(4-hydroxy-6-methoxy-7-methyl-3-oxo-5-phthalanyl)-4-methyl-4-hexanoic acid) is known to reduce the rate of de novo synthesis of guanosine monophosphate by inhibition of inosine monophosphate dehydrogenase (“IMPDH”). It also reduces lymphocyte proliferation.

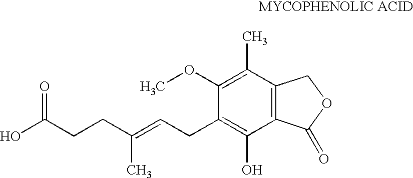

Scientists have shown that mycophenolic acid has a synergistic effect when combined with Abacavir (Ziagen) in vitro. Mycophenolic acid depletes guanosine, one of the essential DNA building blocks. Abacavir is an analog of guanosine and as such, must compete with the body's natural production of guanosine in order to have a therapeutic effect. By depleting naturally occurring guanosine, mycophenolic acid improves Abacavir's uptake by the cell. Scientists have determined that the combination of mycophenolic acid and Abacavir is highly active against Abacavir-resistant virus. However, notably the combination of mycophenolic acid and zidovudine or stavudine was antagonistic, likely due to the inhibition of thymidine phosphorylation by mycophenolic acid. 39th Interscience Conference on Antimicrobial Agents and Chemotherapy, San Francisco, Calif., Sep. 26-29, 1999. Heredia, A., Margolis, D. M., Oldach, D., Hazen, R., Redfield, R. R. (1999) Abacavir in combination with the IMPDH inhibitor mycophenolic acid, is active against multi-drug resistant HIV. J Acquir Immune Defic Syndr.; 22:406-7. Margolis, D. M., Heredia, A., Gaywee, J., Oldach, D., Drusano, G., Redfield, R. R. (1999) Abacavir and mycophenolic acid, an inhibitor of inosine monophosphate dehydrogenase, have profound and synergistic anti-HIV activity. J Acquir Immune Defic Syndr., 21:362-370.

U.S. Pat. No. 4,686,234 describes various derivatives of mycophenolic acid, its synthesis and uses in the treatment of autoimmune disorders, psoriasis, and inflammatory diseases, including, in particular, rheumatoid arthritis, tumors, viruses, and for the treatment of allograft rejection.

On May 5, 1995, Morris et al., in U.S. Pat. No. 5,665,728, disclosed a method of preventing or treating hyperproliferative vascular disease in a mammal by administering an antiproliferative effective amount of rapamycin alone or in combination with mycophenolic acid.

In light of the global threat of the HIV epidemic, it is an object of the present invention to provide new methods and compositions for the treatment of HIV.

It is another object of the present invention to provide methods and compositions to treat drug resistant strains of HIV.

### SUMMARY OF THE INVENTION

It has been unexpectedly found that a drug resistant strain of HIV exhibits the behavior of drug-naive virus when given the combination of a β-D-1,3-dioxolanyl nucleoside and an IMPDH inhibitor. In one nonlimiting embodiment, the HIV strain is resistant to a β-D-1,3-dioxolanyl nucleoside. The present invention, therefore, is directed to compositions and methods for the treatment or prophylaxis of HIV, and in particular to a drug-resistant strain of HIV, including but not limited to a DAPD and/or DXG resistant strain of HIV, in an infected host, and in particular a human, comprising administering an effective amount of a β-D-dioxolanyl purine 1,3-dioxolanyl nucleoside (“β-D-1,3-dioxolanyl nucleosides”) of the formula:

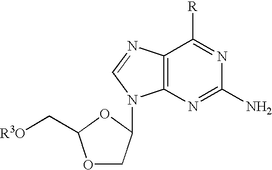

wherein R is H, OH, Cl, NH₂ or NR¹R²; R¹ and R² are independently hydrogen, alkyl or cycloalkyl, and R³ is H, alkyl, aryl, acyl, phosphate, including monophosphate, diphosphate or triphosphate or a stabilized phosphate moiety, including a phospholipid, or an ether-lipid, or its pharmaceutically acceptable salt or prodrug, optionally in a pharmaceutically acceptable carrier or diluent, in combination or alternation with an inosine monophosphate dehydrogenase (IMPDH) inhibitor.

In one embodiment, the enantiomerically enriched β-D-1,3-dioxolanyl purine, and in particular DAPD, is administered in combination or alternation with an IMPDH inhibitor, for example ribavirin, mycophenolic acid, benzamide riboside, tiazofurin, selenazofurin, 5-ethynyl-1-β-D-ribofuranosylimidazole-4-carboxamide (EICAR), or (S)-N-3-[3-(3-methoxy-4-oxazol-5-yl-phenyl)-ureido]-benzyl-carbamic acid tetrahydrofuran-3-yl-ester (VX-497), which effectively decreases the EC₅₀ for DXG when tested against wild type or mutant strains of HIV-1.

In one embodiment, the IMPDH inhibitor is mycophenolic acid. In another preferred embodiment of the invention, the IMPDH inhibitor is ribavirin. In a preferred embodiment, the nucleoside is administered in combination with the IMPDH inhibitor. In a preferred embodiment, the nucleoside is DAPD.

In another embodiment, the enantiomerically enriched β-D-1,3-dioxolanyl purine, and in particular DAPD, is administered in combination or alternation with a compound that reduces the rate of de novo synthesis of guanosine or deoxyguanosine nucleotides.

In a preferred embodiment, DAPD is administered in combination or alternation with ribavirin or mycophenolic acid which reduces the rate of de novo synthesis of guanosine nucleotides.

In yet another embodiment, the enantiomerically enriched β-D-1,3-dioxolanyl purine, and in particular DAPD, is administered in combination or alternation with a compound that effectively increases the intracellular concentration of DXG-TP.

In yet another preferred embodiment, DAPD is administered in combination or alternation with ribavirin or mycophenolic acid that effectively increases the intracellular concentration of DXG-TP.

It has also been discovered that, for example, this drug combination can be used to treat DAPD-resistant and DXG-resistant strains of HIV. DAPD and DXG resistant strains of HIV, after treatment with the disclosed drug combination, exhibit characteristics of drug-naïve virus.

Therefore, in yet another embodiment of the present invention, the enantiomerically enriched β-D-1,3-dioxolanyl purine, and in particular DAPD, is administered in combination or alternation with an IMPDH inhibitor that effectively reverses drug resistance observed in HIV-1 mutant strains.

In yet another embodiment of the present invention, the enantiomerically enriched β-D-1,3-dioxolanyl purine, and in particular DAPD, is administered in combination or alternation with an IMPDH inhibitor that effectively reverses DAPD or DXG drug resistance observed in HIV-1 mutant strains.

In general, during alternation therapy, an effective dosage of each agent is administered serially, whereas in combination therapy, effective dosages of two or more agents are administered together. The dosages will depend on such factors as absorption, bio-distribution, metabolism and excretion rates for each drug as well as other factors known to those of skill in the art. It is to be noted that dosage values will also vary with the severity of the condition to be alleviated. It is to be further understood that for any particular subject, specific dosage regimens and schedules should be adjusted over time according to the individual need and the professional judgment of the person administering or supervising the administration of the compositions. Examples of suitable dosage ranges can be found in the scientific literature and in the Physicians Desk Reference. Many examples of suitable dosage ranges for other compounds described herein are also found in public literature or can be identified using known procedures. These dosage ranges can be modified as desired to achieve a desired result.

The disclosed combination and alternation regiments are useful in the prevention and treatment of HIV infections and other related conditions such as AIDS-related complex (ARC), persistent generalized lymphadenopathy (PGL), AIDS-related neurological conditions, anti-HIV antibody positive and HIV-positive conditions, Kaposi's sarcoma, thrombocytopenia purpurea and opportunistic infections. In addition, these compounds or formulations can be used prophylactically to prevent or retard the progression of clinical illness in individuals who are anti-HIV antibody or HIV-antigen positive or who have been exposed to HIV.

### DETAILED DESCRIPTION OF THE INVENTION

It has been unexpectedly found that a drug resistant strain of HIV exhibits the behavior of drug-naive virus when given the combination of a β-D-1,3-dioxolanyl nucleoside and an IMPDH inhibitor. In one nonlimiting embodiment, the HIV strain is resistant to a β-D-1,3-dioxolanyl nucleoside.

IMPDH catalyzes the NAD-dependent oxidation of inosine-5′-monophosphate (IMP) to xanthosine-5′-monophosphate (XMP), which is a necessary step in guanosine nucleotide synthesis. It has been discovered that reduction of intracellular deoxy-guanosine 5′-triphosphate (dGTP) levels through inhibition of inosine monophosphate dehydrogenase (IMPDH) effectively increases the intracellular concentration of DXG-TP thereby augmenting inhibition HIV replication. This alone, however, cannot explain the unexpected sensitivity of a drug resistant form of HIV to a β-D-1,3-dioxolanyl nucleoside administered in the presence of an IMPDH inhibitor.

Therefore, the present invention is directed to compositions and methods for the treatment or prophylaxis of HIV, and in particular to drug-resistant strains of HIV, such as DAPD and/or DXG resistant strains of HIV, in a host, for example a mammal, and in particular a human, comprising administering an effective amount of an enantiomerically enriched β-D-1,3-dioxolanyl purine of the formula:

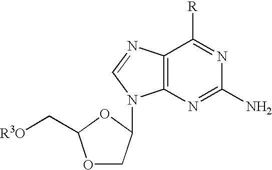

wherein R is H, OH, Cl, NH₂ or NR¹R²; R¹ and R² are independently hydrogen, alkyl or cycloalkyl, and R³ is H, alkyl, aryl, acyl, phosphate, including monophosphate, diphosphate or triphosphate or a stabilized phosphate moiety, including a phospholipid, or an ether-lipid or its pharmaceutically acceptable salt or prodrug, optionally in a pharmaceutically acceptable carrier or diluent, in combination or alternation with an inosine monophosphate dehydrogenase (IMPDH) inhibitor.

In one embodiment, the enantiomerically enriched β-D-1,3-dioxolanyl purine, and in particular DAPD, is administered in combination or alternation with an IMPDH inhibitor, for example ribavirin, mycophenolic acid, benzamide riboside, tiazofurin, selenazofurin, 5-ethynyl-1-β-D-ribofuranosylimidazole-4-carboxamide (EICAR), or (S)-N-3-[3-(3-methoxy-4-oxazol-5-yl-phenyl)-ureido]-benzyl-carbamic acid tetrahydrofuran-3-yl-ester (VX-497), which effectively decreases the EC₅₀ for DXG when tested against wild type or mutant strains of HIV-1.

In a preferred embodiment, the IMPDH inhibitor is mycophenolic acid. In another preferred embodiment of the invention, the IMPDH inhibitor is ribavirin. In a preferred embodiment, the nucleoside is administered in combination with the IMPDH inhibitor. In another preferred embodiment, the nucleoside is DAPD.

In another embodiment, the enantiomerically enriched β-D-1,3-dioxolanyl purine, and in particular DAPD, is administered in combination or alternation with a compound that reduces the rate of de novo synthesis of guanosine and deoxyguanosine nucleotides.

In a preferred embodiment, DAPD is administered in combination or alternation with ribavirin or mycophenolic acid which reduces the rate of de novo synthesis of guanosine nucleotides.

In yet another embodiment, the enantiomerically enriched β-D-1,3-dioxolanyl purine, and in particular DAPD, is administered in combination or alternation with a compound that effectively increases the intracellular concentration of DXG-TP.

In yet another preferred embodiment, DAPD is administered in combination or alternation with ribavirin or mycophenolic acid that effectively increases the intracellular concentration of DXG-TP.

It has also been discovered that, for example, this drug combination can be used to treat DAPD-resistant and DXG-resistant strains of HIV. DAPD and DXG resistant strains of HIV, after treatment with the disclosed drug combination, exhibit characteristics of drug-naive virus.

Therefore, in yet another embodiment of the present invention, the enantiomerically enriched β-D-1,3-dioxolanyl purine, and in particular DAPD, is administered in combination or alternation with an IMPDH inhibitor that effectively reverses drug resistance observed in HIV-1 mutant strains.

In yet another embodiment of the present invention, the enantiomerically enriched β-D-1,3-dioxolanyl purine, and in particular DAPD, is administered in combination or alternation with an IMPDH inhibitor that effectively reverses DAPD or DXG drug resistance observed in HIV-1 mutant strains.

I. Definitions

The term “protected” as used herein and unless otherwise defined refers to a group that is added to an oxygen, nitrogen, or phosphorus atom to prevent its further reaction or for other purposes. A wide variety of oxygen and nitrogen protecting groups are known to those skilled in the art of organic synthesis.

The term halo, as used herein, includes chloro, bromo, iodo and fluoro.

The term alkyl, as used herein, unless otherwise specified, refers to a saturated straight, branched, or cyclic, primary, secondary or tertiary hydrocarbon of typically C₁ to C₁₀, and specifically includes methyl, trifluoromethyl, ethyl, propyl, isopropyl, cyclopropyl, butyl, isobutyl, t-butyl, pentyl, cyclopentyl, isopentyl, neopentyl, hexyl, isohexyl, cyclohexyl, cyclohexylmethyl, 3-methylpentyl, 2,2-dimethylbutyl, and 2,3-dimethylbutyl. The term includes both substituted and unsubstituted alkyl groups. Moieties with which the alkyl group can be substituted are selected from the group consisting of hydroxyl, amino, alkylamino, arylamino, alkoxy, aryloxy, nitro, cyano, sulfonic acid, sulfate, phosphonic acid, phosphate, or phosphonate, either unprotected, or protected as necessary, as known to those skilled in the art, for example, as taught in Greene, et al., Protective Groups in Organic Synthesis, John Wiley and Sons, Second Edition, 1991, hereby incorporated by reference.

The term lower alkyl, as used herein, and unless otherwise specified, refers to a C₁ to C₄ saturated straight, branched, or if appropriate, a cyclic (for example, cyclopropyl) alkyl group, including both substituted and unsubstituted forms. Unless otherwise specifically stated in this application, when alkyl is a suitable moiety, lower alkyl is preferred. Similarly, when alkyl or lower alkyl is a suitable moiety, unsubstituted alkyl or lower alkyl is preferred.

The term aryl, as used herein, and unless otherwise specified, refers to phenyl, biphenyl, or naphthyl, and preferably phenyl. The term includes both substituted and unsubstituted moieties. The aryl group can be substituted with one or more moieties selected from the group consisting of hydroxyl, amino, alkylamino, arylamino, alkoxy, aryloxy, nitro, cyano, sulfonic acid, sulfate, phosphonic acid, phosphate, or phosphonate, either unprotected, or protected as necessary, as known to those skilled in the art, for example, as taught in Greene, et al., Protective Groups in Organic Synthesis, John Wiley and Sons, Second Edition, 1991.

The term acyl refers to a carboxylic acid ester in which the non-carbonyl moiety of the ester group is selected from straight, branched, or cyclic alkyl or lower alkyl, alkoxyalkyl including methoxymethyl, aralkyl including benzyl, aryloxyalkyl such as phenoxymethyl, aryl including phenyl optionally substituted with halogen (e.g., F, Cl, Br or I), C₁, to C₄ alkyl or C₁ to C₄ alkoxy, sulfonate esters such as alkyl or aralkyl sulphonyl including methanesulfonyl, the mono, di or triphosphate ester, trityl or monomethoxytrityl, substituted benzyl, trialkylsilyl (e.g. dimethyl-t-butylsilyl) or diphenylmethylsilyl. Aryl groups in the esters optimally comprise a phenyl group. The term “lower acyl” refers to an acyl group in which the non-carbonyl moiety is lower alkyl.

The term “enantiomerically enriched” is used throughout the specification to describe a compound which includes approximately 95% or greater, preferably at least 96%, more preferably at least 97%, even more preferably, at least 98%, and even more preferably at least about 99% or more of a single enantiomer of that compound. When a nucleoside of a particular configuration (D or L) is referred to in this specification, it is presumed that the nucleoside is an enantiomerically enriched nucleoside, unless otherwise stated.

The term “host,” as used herein, refers to a unicellular or multicellular organism in which the virus can replicate, including cell lines and animals, and preferably a human. Alternatively, the host can be carrying a part of the viral genome, whose replication or function can be altered by the compounds of the present invention. The term host specifically refers to infected cells, cells transfected with all or part of the viral genome and animals, in particular, primates (including chimpanzees) and humans. In most animal applications of the present invention, the host is a human patient. Veterinary applications, in certain indications, however, are clearly anticipated by the present invention (such as simian immunodeficiency virus in chimpanzees).

Pharmaceutically acceptable prodrugs refer to a compound that is metabolized, for example hydrolyzed or oxidized, in the host to form the compound of the present invention. Typical examples of prodrugs include compounds that have biologically labile protecting groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, dephosphorylated to produce the active compound. Pharmaceutically acceptable salts include those derived from pharmaceutically acceptable inorganic or organic bases and acids. Suitable salts include those derived from alkali metals such as potassium and sodium, alkaline earth metals such as calcium and magnesium, among numerous other acids well known in the pharmaceutical art. The compounds of this invention either possess antiviral activity, or are metabolized to a compound that exhibits such activity.

II. Pharmaceutically Acceptable Salts and Prodrugs

In cases where any of the compounds as disclosed herein are sufficiently basic or acidic to form stable nontoxic acid or base salts, administration of the compound as a pharmaceutically acceptable salt may be appropriate. Examples of pharmaceutically acceptable salts are organic acid addition salts formed with acids, which form a physiological acceptable anion, for example, tosylate, methanesulfonate, acetate, citrate, malonate, tartarate, succinate, benzoate, ascorbate, α-ketoglutarate and α-glycerophosphate. Suitable inorganic salts may also be formed, including, sulfate, nitrate, bicarbonate and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

Any of the nucleosides described herein can be administered as a nucleotide prodrug to increase the activity, bioavailability, stability or otherwise alter the properties of the nucleoside. A number of nucleotide prodrug ligands are known. In general, alkylation, acylation or other lipophilic modification of the hydroxyl group of the compound or of the mono, di or triphosphate of the nucleoside will increase the stability of the nucleotide. Examples of substituent groups that can replace one or more hydrogens on the phosphate moiety are alkyl, aryl, steroids, carbohydrates, including sugars, 1 ,2-diacylglycerol and alcohols. Many are described in R. Jones and N. Bischofberger, Antiviral Research, 27 (1995) 1-17. Any of these can be used in combination with the disclosed nucleosides to achieve a desired effect.

Any of the compounds which are described herein for use in combination or alternation therapy can be administered as an acylated prodrug, wherein the term acyl refers to a carboxylic acid ester in which the non-carbonyl moiety of the ester group is selected from straight, branched, or cyclic alkyl or lower alkyl, alkoxyalkyl including methoxymethyl, aralkyl including benzyl, aryloxyalkyl such as phenoxymethyl, aryl including phenyl optionally substituted with halogen, C₁ to C₄ alkyl or C₁ to C₄ alkoxy, sulfonate esters such as alkyl or aralkyl sulphonyl including methanesulfonyl, the mono, di or triphosphate ester, trityl or monomethoxytrityl, substituted benzyl, trialkylsilyl (e.g. dimethyl-t-butylsilyl).

The active nucleoside or other hydroxyl containing compound can also be provided as an ether lipid (and particularly a 5′-ether lipid or a 5′-phosphoether lipid for a nucleoside), as disclosed in the following references, which are incorporated by reference herein: Kucera, L. S., N. Iyer, E. Leake, A. Raben, Modest E. K., D. L. W., and C. Piantadosi. 1990. “Novel membrane-interactive ether lipid analogs that inhibit infectious HIV-1 production and induce defective virus formation.” AIDS Res. Hum. Retro Viruses. 6:491-501; Piantadosi, C., J. Marasco C. J., S. L. Morris-Natschke, K. L. Meyer, F. Gumus, J. R. Surles, K. S. Ishaq, L. S. Kucera, N. Iyer, C. A. Wallen, S. Piantadosi, and E. J. Modest. 1991. “Synthesis and evaluation of novel ether lipid nucleoside conjugates for anti-HIV activity.” J. Med. Chem. 34:1408.1414; Hosteller, K. Y., D. D. Richman, D. A. Carson, L. M. Stuhmiller, G. M. T. van Wijk, and H. van den Bosch. 1992. “Greatly enhanced inhibition of human immunodeficiency virus type 1 replication in CEM and HT4-6C cells by 3′-deoxythymidine diphosphate dimyristoylglycerol, a lipid prodrug of 3,-deoxythymidine.” Antimicrob. Agents Chemother. 36:2025.2029; Hostetler, K. Y., L. M. Stuhmiller, H. B. Lenting, H. van den Bosch, and D. D. Richman, 1990. “Synthesis and antiretroviral activity of phospholipid analogs of azidothymidine and other antiviral nucleosides.” J. Biol. Chem. 265:61127.

Nonlimiting examples of U.S. patents that disclose suitable lipophilic substituents that can be covalently incorporated into the nucleoside or other hydroxyl or amine containing compound, preferably at the 5′-OH position of the nucleoside or lipophilic preparations, include U.S. Pat. Nos. 5,149,794 (Sep. 22, 1992, Yatvin et al.); 5,194,654 (Mar. 16, 1993, Hostetler et al., 5,223,263 (Jun. 29, 1993, Hostetler et al.); 5,256,641 (Oct. 26, 1993, Yatvin et al.); 5,411,947 (May 2, 1995, Hostetler et al.); 5,463,092 (Oct. 31, 1995, Hostetler et al.); 5,543,389 (Aug. 6, 1996, Yatvin et al.); 5,543,390 (Aug. 6, 1996, Yatvin et al.); 5,543,391 (Aug. 6, 1996, Yatvin et al.); and 5,554,728 (Sep. 10, 1996; Basava et al.), all of which are incorporated herein by reference. Foreign patent applications that disclose lipophilic substituents that can be attached to the nucleosides of the present invention, or lipophilic preparations, include WO 89/02733, WO 90/00555, WO 91/16920, WO 91/18914, WO 93/00910, WO 94/26273, WO 96/15132, EP 0 350 287, EP 93917054.4, and WO 91/19721.

Nonlimiting examples of nucleotide prodrugs are described in the following references: Ho, D. H. W. (1973) “Distribution of Kinase and deaminase of 1β-D-arabinofuranosylcytosine in tissues of man and muse.”Cancer Res. 33, 2816-2820; Holy, A. (1993) Isopolar phosphorous-modified nucleotide analogues,” In: De Clercq (Ed.), Advances in Antiviral Drug Design, Vol. I, JAI Press, pp. 179-231; Hong, C. I., Nechaev, A., and West, C.R. (1979a) “Synthesis and antitumor activity of 1-β-D-arabino-furanosylcytosine conjugates of cortisol and cortisone.” Bicohem. Biophys. Rs. Commun. 88, 1223-1229; Hong, C. I., Nechaev, A., Kirisits, A. J. Buchheit, D. J. and West, C. R. (1980) “Nucleoside conjugates as potential antitumor agents. 3. Synthesis and antitumor activity of 1-(β-D-arabinofuranosyl) cytosine conjugates of corticosteriods and selected lipophilic alcohols.” J. Med. Chem. 28, 171-177; Hosteller, K. Y., Stuhmiller, L. M., Lenting, H. B. M. van den Bosch, H. and Richman J. Biol. Chem. 265, 6112-6117; Hosteller, K. Y., Carson, D. A. and Richman, D. D. (1991); “Phosphatidylazidothymidine: mechanism of antiretroviral action in CEM cells.” J. Biol Chem. 266, 11714-11717; Hosteller, K. Y., Korba, B. Sridhar, C., Gardener, M. (1994a) “Antiviral activity of phosphatidyl-dideoxycytidine in hepatitis B-infected cells and enhanced hepatic uptake in mice.” Antiviral Res. 24, 59-67; Hosteller, K. Y., Richman, D. D., Sridhar. C. N. Felgner, P. L. Felgner, J., Ricci, J., Gardener, M. F. Selleseth, D. W. and Ellis, M. N. (1994b) “Phosphatidylazidothymidine and phosphatidyl-ddC: Assessment of uptake in mouse lymphoid tissues and antiviral activities in human immunodeficiency virus-infected cells and in rauscher leukemia virus-infected mice.” Antimicrobial Agents Chemother. 38, 2792-2797; Hunston, R. N., Jones, A. A. McGuigan, C., Walker, R. T., Balzarini, J., and DeClercq, E. (1984) “Synthesis and biological properties of some cyclic phosphotriesters derived from 2′-deoxy-5-fluorouridine.” J. Med. Chem. 27, 440-444; Ji, Y. H., Moog, C., Schmitt, G., Bischoff, P. and Luu, B. (1990); “Monophosphoric acid esters of 7-β-hydroxycholesterol and of pyrimidine nucleoside as potential antitumor agents: synthesis and preliminary evaluation of antitumor activity.” J. Med. Chem. 33 2264-2270; Jones, A. S., McGuigan, C., Walker, R. T., Balzarini, J. and DeClercq, E. (1984) “Synthesis, properties, and biological activity of some nucleoside cyclic phosphoramidates.” J. Chem. Soc. Perkin Trans. I, 1471-1474; Juodka, B. A. and Smrt, J. (1974) “Synthesis of diribonucleoside phosph (P→N) amino acid derivatives.” Coll. Czech. Chem. Comm. 39, 363-968; Kataoka, S., Imai, J., Yamaji, N., Kato, M., Saito, M., Kawada, T. and Imai, S. (1989) “Alkylated cAMP derivatives; selective synthesis and biological activities.” Nucleic Acids Res. Sym. Ser. 21, 1-2; Kataoka, S., Uchida, “(cAMP) benzyl and methyl triesters.” Heterocycles 32, 1351-1356; Kinchington, D., Harvey, J. J., O'Connor, T.J., Jones, B. C. N. M., Devine, K. G., Taylor-Robinson D., Jeffries, D. J. and McGuigan, C. (1992) “Comparison of antiviral effects of zidovudine phosphoramidate and phosphorodiamidate derivatives against HIV and ULV in vitro.” Antiviral Chem. Chemother. 3, 107-112; Kodama, K., Morozumi, M., Saithoh, K. I., Kuninaka, H., Yosino, H. and Saneyoshi, M. (1989) “Antitumor activity and pharmacology of 1-β-D-arabinofuranosylcytosine −5′-stearylphosphate; an orally active derivative of 1-β-D-arabinofuranosylcytosine.” Jpn. J. Cancer Res. 80, 679-685; Korty, M. and Engels, J. (1979) “The effects of adenosine- and guanosine 3′,5′ phosphoric and acid benzyl esters on guinea-pig ventricular myocardium.” Naunyn-Schmiedeberg's Arch. Pharmacol. 310, 103-111; Kumar, A., Goe, P. L., Jones, A. S. Walker, R. T. Balzarini, J. and DeClercq, E. (1990) “Synthesis and biological evaluation of some cyclic phosphoramidate nucleoside derivatives.” J. Med. Chem, 33, 2368-2375; LeBec, C., and Huynh-Dinh, T. (1991) “Synthesis of lipophilic phosphate triester derivatives of 5-fluorouridine an arabinocytidine as anticancer prodrugs.” Tetrahedron Lett. 32, 6553-6556; Lichtenstein, J., Barner, H. D. and Cohen, S. S. (1960) “The metabolism of exogenously supplied nucleotides by Escherichia coli.,” J. Biol. Chem. 235, 457-465; Lucthy, J., Von Daeniken, A., Friederich, J. Manthey, B., Zweifel, J., Schlatter, C. and Benn, M. H. (1981) “Synthesis and toxicological properties of three naturally occurring cyanoepithioalkanes”. Mitt. Geg. Lebensmittelunters. Hyg. 72, 131-133 (Chem. Abstr. 95, 127093); McGigan, C. Tollerfield, S. M. and Riley, P. a. (1989) “Synthesis and biological evaluation of some phosphate triester derivatives of the anti-viral drug Ara.” Nucleic Acids Res. 17, 6065-6075; McGuigan, C., Devine, K. G., O'Connor, T. J., Galpin, S. A., Jeffries, D. J. and Kinchington, D. (1990a) “Synthesis and evaluation of some novel phosphoramidate derivatives of 3 ′-azido-3 ′-deoxythymidine (AZT) as anti-HIV compounds.” Antiviral Chem. Chemother. 107-113; McGuigan, C., O'Connor, T. J., Nicholls, S. R. Nickson, C. and Kinchington, D. (1990b) “Synthesis and anti-HIV activity of some novel substituted dialkyl phosphate derivatives of AZT and ddCyd.” Antiviral Chem. Chemother. 1, 355-360; McGuigan, C., Nicholls, S.R., O'Connor, T. J., and Kinchington, D. (1990c) “Synthesis of some novel dialkyl phosphate derivative of 3′-modified nucleosides as potential anti-AIDS drugs.” Antiviral Chem. Chemother. 1, 25-33; McGuigan, C., Devin, K. G., O'Connor, T. J., and Kinchington, D. (1991) “Synthesis and anti-HIV activity of some haloalkyl phosphoramidate derivatives of 3′-azido-3′-deoxythymidine (AZT); potent activity of the trichloroethyl methoxyalaninyl compound.” Antiviral Res. 15, 255-263; McGuigan, C., Pathirana, R. N., Balzarini, J. and DeClercq, E. (1993b) “Intracellular delivery of bioactive AZT nucleotides by aryl phosphate derivatives of AZT.” J. Med. Chem. 36, 1048-1052.

Alkyl hydrogen phosphate derivatives of the anti-HIV agent AZT may be less toxic than the parent nucleoside analogue. Antiviral Chem. Chemother. 5, 271-277; Meyer, R. B., Jr., Shuman, D. A. and Robins, R. K. (1973) “Synthesis of purine nucleoside 3′, 5′-cyclic phosphoramidates.” Tetrahedron Lett. 269-272; Nagyvary, J. Gohil, R. N., Kirchner, C. R. and Stevens, J. D. (1973) “Studies on neutral esters of cyclic AMP,” BioChem. Biophys. Res. Commun. 55, 1072-1077; Namane, A. Gouyette, C., Fillion, M. P., Fillion, G. and Huynh-Dinh, T. (1992) “Improved brain delivery of AZT using a glycosyl phosphotriester prodrug.” J. Med. Chem. 35, 3039-3044; Nargeot, J. Nerbonne, J. M. Engels, J. and Leser, H. A. (1983) Natl. Acad. Sci. U.S.A. 80, 2395-2399; Nelson, K. A., Bentrude, W. G. Stser, W. N. and Hutchinson, J. P. (1987) “The question of chair-twist equilibria for the phosphate rings of nucleoside cyclic 3′, 5′ monophosphates. ¹HNMR and x-ray crystallographic study of the diastereomers of thymidine phenyl cyclic 3′, 5′-monophosphate.” J. Am. Chem. Soc. 109, 4058-4064; Nerbonne, J. M., Richard, S., Nargeot, J. and Lester, H. A. (1984) “New photoactivatable cyclic nucleotides produce intracellular jumps in cyclic AMP and cyclic GMP concentrations.” Nature 301, 74-76; Neumann, J. M., Herv_, M., Debouzy, J. C., Guerra, F. I., Gouyette, C., Dupraz, B. and Huyny-Dinh, T. (1989) “Synthesis and transmembrane transport studies by NMR of a glycosyl phospholipid of thymidine.” J. Am. Chem. Soc. 111, 4270-4277; Ohno, R., Tatsumi, N., Hirano, M., Imai, K. Mizoguchi, H., Nakamura, T., Kosaka, M., Takatuski, K., Yamaya, T., Toyama K., Yoshida, T., Masaoka, T., Hashimoto, S., Ohshima, T., Kimura, I., Yamada, K. and Kimura, J. (1991) “Treatment of myelodysplastic syndromes with orally administered 1-β-D-arabinouranosylcytosine -5′ stearylphosphate.” Oncology 48, 451-455. Palomino, E., Kessle, D. and Horwitz, J. P. (1989) “A dihydropyridine carrier system for sustained delivery of 2′, 3′ dideoxynucleosides to the brain.” J. Med. Chem. 32, 22-625; Perkins, R. M., Barney, S. Wittrock, R., Clark, P. H., Levin, R. Lambert, D. M., Petteway, S. R., Serafinowska, H. T., Bailey, S. M., Jackson, S., Hamden, M. R. Ashton, R., Sutton, D., Harvey, J. J. and Brown, A. G. (1993) “Activity of BRL47923 and its oral prodrug, SB203657A against a rauscher murine leukemia virus infection in mice.” Antiviral Res. 20 (Suppl. I). 84; Piantadosi, C., Marasco, C. J., Jr., Norris-Natschke, S. L., Meyer, K. L., Gumus, F., Surles, J. R., Ishaq, K. S., Kucera, L. S. Iyer, N., Wallen, C. A., Piantadosi, S. and Modest, E. J. (1991) “Synthesis and evaluation of novel ether lipid nucleoside conjugates for anti-HIV-1 activity.” J. Med. Chem. 34, 1408-1414; Pompon, A., Lefebvre, I., Imbach, J. L., Kahn, S. and Farquhar, D. (1994). “Decomposition pathways of the mono- and bis(pivaloyloxymethyl) esters of azidothymidine-5′-monophosphate in cell extract and in tissue culture medium; an application of the “on-line ISRβ-cleaning HPLC technique.” Antiviral Chem Chemother. 5, 91-98; Postemark, T. (1974) “Cyclic AMP and cyclic GMP.” Annu. Rev. Pharmacol. 14, 23-33; Prisbe, E. J., Martin, J. C. M., McGhee, D. P. C., Barker, M. F., Smee, D. F. Duke, A. E., Matthews, T. R. and Verheyden, J. P. J. (1986) “Synthesis and antiherpes virus activity of phosphate an phosphonate derivatives of 9-[(1,3-dihydroxy-2-propoxy)methyl] guanine.” J. Med. Chem. 29, 671-675; Pucch, F., Gosselin, G., Lefebvre, I., Pompon, a., Aubertin, A. M. Dim, and Imbach, J. L. (1993) “Intracellular delivery of nucleoside monophosphate through a reductase-mediated activation process.” Antiviral Res. 22, 155-174; Pugaeva, V. P., Klochkeva, S. I., Mashbits, F. D. and Eizengart, R. S. (1969). “Toxicological assessment and health standard ratings for ethylene sulfide in the industrial atmosphere.” Gig. Trf. Prof. Zabol. 14, 47-48 (Chem. Abstr. 72, 212); Robins, R. K. (1984) “The potential of nucleotide analogs as inhibitors of Retro viruses and tumors.” Pharm. Res. 11-18; Rosowsky, A., Kim. S. H., Ross and J. Wick, M. M. (1982) “Lipophilic 5′-(alkylphosphate) esters of 1-1-β-D-arabinofuranosylcytosine and its N⁴-acyl and 2.2′-anhydro-3′-O-acyl derivatives as potential prodrugs.” J. Med. Chem. 25, 171-178; Ross, W. (1961) “Increased sensitivity of the walker turnout towards aromatic nitrogen mustards carrying basic side chains following glucose pretreatment.” BioChem. Pharm. 8, 235-240; Ryu, E. K., Ross, R. J. Matsushita, T., MacCoss, M., Hong, C. I. and West, C. R. (1982). “Phospholipid-nucleoside conjugates. 3. Synthesis and preliminary biological evaluation of 1-β-D-arabinofuranosylcytosine 5′ diphosphate [−], 2-diacylglycerols.” J. Med. Chem. 25, 1322-1329; Saffhill, R. and Hume, W. J. (1986) “The degradation of 5-iododeoxyuridine and 5-bromoethoxyuridine by serum from different sources and its consequences for the use of these compounds for incorporation into DNA.” Chem. Biol. Interact. 57, 347-355; Saneyoshi, M., Morozumi, M., Kodama, K., Machida, J., Kuninaka, A. and Yoshino, H. (1980) “Synthetic nucleosides and nucleotides. XVI. Synthesis and biological evaluations of a series of 1-β-D-arabinofuranosylcytosine 5′-alkyl or arylphosphates.” Chem Pharm. Bull. 28, 2915-2923; Sastry, J. K., Nehete, P. N., Khan, S., Nowak, B. J., Plunkett, W., Arlinghaus, R. B. and Farquhar, D. (1992) “Membrane-permeable dideoxyuridine 5′-monophosphate analogue inhibits human immunodeficiency virus infection.” Mol. Pharmacol. 41, 441-445; Shaw, J. P., Jones, R. J. Arimilli, M. N., Louie, M. S., Lee, W. A. and Cundy, K. C. (1994) “Oral bioavailability of PMEA from PMEA prodrugs in male Sprague-Dawley rats.” 9th Annual AAPS Meeting. San Diego, Calif. (Abstract). Shuto, S., Ueda, S., Imamura, S., Fukukawa, K. Matsuda, A. and Ueda, T. (1987) “A facile one-step synthesis of 5′ phosphatidiylnucleosides by an enzymatic two-phase reaction.” Tetrahedron Lett. 28, 199-202; Shuto, S. Itoh, H., Ueda, S., Imamura, S., Kukukawa, K., Tsujino, M., Matsuda, A. and Ueda, T. (1988) Pharm. Bull. 36, 209-217. An example of a useful phosphate prodrug group is the S-acyl-2-thioethyl group, also referred to as “SATE”.

III. Pharmaceutical Compositions

Humans suffering from effects caused by any of the diseases described herein, and in particular, an infection caused by a drug resistant strain of HIV, can be treated by administering to the patient an effective amount of the defined β-D-1,3-dioxolanyl nucleoside, and in particular, DAPD or DXG, in combination or alternation with an IMPDH inhibitor, including ribavirin or mycophenolic acid, or a pharmaceutically acceptable salt or ester thereof in the presence of a pharmaceutically acceptable carrier or diluent. The active materials can be administered by any appropriate route, for example, orally, parenterally, enterally, intravenously, intradermally, subcutaneously, topically, nasally, rectally, in liquid, or solid form.

The active compounds are included in the pharmaceutically acceptable carrier or diluent in an amount sufficient to deliver to a patient a therapeutically effective amount of compound to inhibit viral replication in vivo, especially HIV replication, without causing serious toxic effects in the treated patient. By “inhibitory amount” is meant an amount of active ingredient sufficient to exert an inhibitory effect as measured by, for example, an assay such as the ones described herein.

A preferred dose of the compound for all the above-mentioned conditions will be in the range from about 1 to 50 mg/kg, preferably 1 to 20 mg/kg, of body weight per day, more generally 0.1 to about 100 mg per kilogram body weight of the recipient per day. The effective dosage range of the pharmaceutically acceptable derivatives can be calculated based on the weight of the parent nucleoside to be delivered. If the derivative exhibits activity in itself, the effective dosage can be estimated as above using the weight of the derivative, or by other means known to those skilled in the art.

The compounds are conveniently administered in unit any suitable dosage form, including but not limited to one containing 7 to 3000 mg, preferably 70 to 1400 mg of active ingredient per unit dosage form. An oral dosage of 50 to 1000 mg is usually convenient.

Ideally, at least one of the active ingredients, though preferably the combination of active ingredients, should be administered to achieve peak plasma concentrations of the active compound of from about 0.2 to 70 mM, preferably about 1.0 to 10 mM. This may be achieved, for example, by the intravenous injection of a 0.1 to 10% solution of the active ingredient, optionally in saline, or administered as a bolus of the active ingredient.

The concentration of active compound in the drug composition will depend on absorption, distribution, metabolism and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient may be administered at once, or may be divided into a number of smaller doses to be administered at varying intervals of time.

A preferred mode of administration of the active compound is oral. Oral compositions will generally include an inert diluent or an edible carrier. They may be enclosed in gelatin capsules or compressed into tablets. For the purpose of oral therapeutic administration, the active compound can be incorporated with excipients and used in the form of tablets, troches, or capsules. Pharmaceutically compatible bind agents, and/or adjuvant materials can be included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or other enteric agents.

The compounds can be administered as a component of an elixir, suspension, syrup, wafer, chewing gum or the like. A syrup may contain, in addition to the active compounds, sucrose as a sweetening agent and certain preservatives, dyes and colorings and flavors.

The compounds or their pharmaceutically acceptable derivative or salts thereof can also be mixed with other active materials that do not impair the desired action, or with materials that supplement the desired action, such as antibiotics, anti-fungals, anti-inflammatories, protease inhibitors, or other nucleoside or non-nucleoside antiviral agents, as discussed in more detail above. Solutions or suspensions used for parental, intradermal, subcutaneous, or topical application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parental preparation can be enclosed in ampoules, disposable syringes or multiple dose vials made of glass or plastic.

If administered intravenously, preferred carriers are physiological saline or phosphate buffered saline (PBS).

If administered by nasal aerosol or inhalation, these compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

If rectally administered in the form of suppositories, these compositions may be prepared by mixing the drug with a suitable non-initiating excipient, such as cocoa butter, synthetic glyceride esters of polyethylene glycols, which are solid at ordinary temperatures, but liquefy and/or dissolve in the rectal cavity to release the drug.

In a preferred embodiment, the active compounds are prepared with carriers that will protect the compound against rapid elimination from the body, such as a controlled release formulation, including implants and micro-encapsulated delivery systems. Biodegradable, biocompatible polymers can be used, such as ethylene vinyl acetate, polyanhydrides, polyglycolic acid, collagen, polyorthoesters and polylactic acid. Methods for preparation of such formulations will be apparent to those skilled in the art. The materials can also be obtained commercially from Alza Corporation.

Liposomal suspensions (including liposomes targeted to infected cells with monoclonal antibodies to viral antigens) are also preferred as pharmaceutically acceptable carriers. these may be prepared according to methods known to those skilled in the art, for example, as described in U.S. Pat. No. 4,522,811 (which is incorporated herein by reference in its entirety). For example, liposome formulations may be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the active compound or its monophosphate, diphosphate, and/or triphosphate derivatives is then introduced into the container. The container is then swirled by hand to free lipid material from the sides of the container and to disperse lipid aggregates, thereby forming the liposomal suspension.

IV. Combination and Alternation Therapies for the Treatment of HIV Infection

In general, during alternation therapy, an effective dosage of each agent is administered serially, whereas in combination therapy, effective dosages of two or more agents are administered together. The dosages will depend on such factors as absorption, bio-distribution, metabolism and excretion rates for each drug as well as other factors known to those of skill in the art. It is to be noted that dosage values will also vary with the severity of the condition to be alleviated. It is to be further understood that for any particular subject, specific dosage regimens and schedules should be adjusted over time according to the individual need and the professional judgment of the person administering or supervising the administration of the compositions. Examples of suitable dosage ranges can be found in the scientific literature and in the Physicians Desk Reference. Many examples of suitable dosage ranges for other compounds described herein are also found in public literature or can be identified using known procedures. These dosage ranges can be modified as desired to achieve a desired result.

The disclosed combination and alternation regiments are useful in the prevention and treatment of HIV infections and other related conditions such as AIDS-related complex (ARC), persistent generalized lymphadenopathy (PGL), AIDS-related neurological conditions, anti-HIV antibody positive and HIV-positive conditions, Kaposi's sarcoma, thrombocytopenia purpurea and opportunistic infections. In addition, these compounds or formulations can be used prophylactically to prevent or retard the progression of clinical illness in individuals who are anti-HIV antibody or HIV-antigen positive or who have been exposed to HIV.

It has been discovered that, for example, this drug combination can be used to treat DAPD-resistant and DXG-resistant strains of HIV. DAPD and DXG resistant strains of HIV, after treatment with the disclosed drug combination, exhibit characteristics of drug-naïve virus.

In addition, compounds according to the present invention can be administered in combination or alternation with one or more antiviral, anti-HBV, anti-HCV or anti-herpetic agent or interferon, anti-cancer, antiproliferative or antibacterial agents, including other compounds of the present invention. Certain compounds according to the present invention may be effective for enhancing the biological activity of certain agents according to the present invention by reducing the metabolism, catabolism or inactivation of other compounds and as such, are co-administered for this intended effect.

Illustrative and nonlimiting examples of the present invention are provided below. These examples are not intended to limit the scope of the invention.

V. Ribavirin in Combination with DAPD

Ribavirin (RBV) was analyzed in vitro for activity against HIV-1 and for its effects on the in vitro anti-HIV activity of two dGTP analogues, DAPD and DXG. RBV was also evaluated for cytotoxicity in the laboratory adapted cell line MT2 and in peripheral blood mononuclear cells (PBMC). RBV is an inhibitor of the enzyme IMP dehydrogenase. This enzyme is part of the pathway utilized by cells for the de novo synthesis of GTP.

Cytotoxicity Assays:

RBV was tested for cytotoxicity on the laboratory adapted T-cell line MT2 and in PBMCs using a XTT based assay. The XTT (2,3-bis(2-methoxy-4-nitro-5-sulfoxyphenyl)-5[(phenylamino)carbonyl]-2H-tetrazolium hydroxide) assay is an in vitro colorimetric cyto-protection assay. Reduction of XTT by mitochondria dehydrogenases results in the cleavage of the tetrazolium ring of XTT, yielding orange formazan crystals, which are soluble in aqueous solution. The resultant orange solution was read in a spectrophotometer at a wavelength of 450nM. RBV was prepared in 100% DMSO at a final concentration of 100 mM. For the cytotoxicity assays, a 2 mM solution of RBV was prepared in cell culture media (RPMI supplemented with 10% fetal calf serum, L-Glutamine 1 mg/ml and 20 ug/ml gentamicin) followed by 2 fold serial dilutions on a 96 well plate. Cells were added to the plat at 3×10⁴/well (MTX) and 2×10⁵/well (PBMC) and the plates were incubated for 5 days at 37° C. in a 5% CO₂ incubator (addition of the cells to the plate diluted the compound to a final high concentration of 1 mM). At the end of the 5-day incubation, XTT was added to each well and incubated at 37° C. for 3 hours followed by the addition of acidified isopropanol. The plate was read at 450 nm in a 96 well plate reader. A dose response curve was generated using the absorption values of cells grown in the absence of compound as 100% protection.

RBV was not toxic in these assays at concentration of up to 1 mM, Table 1.

TABLE 1
Cytotoxicity of RBV

| Cell Type | CC₅₀ |
|---|---|
| MT2 | >1 mM |
| PBMC | >1 mM |

Sensitivity Assays

XXT Assay

RBV was tested for activity against the xxLAI strain of HIV-1 in the laboratory adapted cell line MT2. Dilutions of RBV were made in cell culture media in a 96 well plate; the highest concentration tested was 100 μM. Triplicate samples of compound were tested. MT2 cells were infected with xxLAI at a multiplicity of infection (MOI) of 0.03 for 3 hours at 37° C. in 5% CO₂. The infected cells were plated at 3.0×10⁴/well into a 96 well plated containing drug dilutions and incubated for 5 days at 37° C. in CO₂. The antiviral activity of RBV was determined using the XTT assay described above. This method has been modified into a susceptibility assay and has been used in a variety of in vitro antiviral tests and is readily adaptable to any system with a lytic virus (Weislow, O. S., et. al. 1989). Using the absorption values of the cell controls as 100% protection and no drug, virus infected cells as 0% protection, a dose response curve is generated by plotting % protection on the Y axis and drug concentration on the X axis. From this curve EC₅₀ values were determined.

RBV was not active against HIV-1 in these assays at any of the concentrations tested.

P24 Assay

RBV was also tested for activity against the xxLAI strain of HIV-1 in PBMCs using a p24 based ELISA assay. In this assay, cell supernatants were incubated on microelisa wells coated with antibodies to HIV-1 p24 core antigen. Subsequently, anti-HIV-1 conjugate labeled with horseradish peroxidase was added. The labeled antibody bound to the solid phase antibody/antigen complexes previously formed. Addition of the tetramethylbenzidine substrate results in blue color formation. The color turned yellow when the reaction is stopped. The plates were then analyzed on a plate reader set at 490 nm. The absorbance is a direct measurement of the amount of HIV-1 produced in each well and a decrease in color indicates decreased viral production. Dilutions of RBV were made in cell culture media in a 96 well plate, the highest concentration of RBV tested was 100 μM. PBMC were obtained from HIV-1 negative donors by banding on Ficoll gradients, stimulated with phytohemaglutinin (PHAP) for 48 hours prior to infection with HIV-1, and infected with virus for 4 hours at 37° C. at a MOI of 0.001. Infected cells were seeded into 96 well plates containing 5-fold serial dilutions of RBV. Plates were incubated for 3 days at 37° C. The concentration of virus in each well was determined using the NEN p24 assay. Using the absorption values of the cell controls as 100% protection and drug free, virus infected cells as 0% protection, a dose response curve is generated by plotting percent protection on the Y axis and drug concentration on the X axis. From this curve, EC₅₀ values were determined.

RBV inhibited HIV-1 replication in PBMCs with a median EC₅₀ of 20.5 μM ±11.8.

Combination Assays

The effects of RBV on the in vitro anti-HIV-1 activity of DAPD and DXG were evaluated using the MT2/XTT and PBMC/p24 assays described above. The effects of RBV on the activity of Abacavir and AZT were also analyzed.

MT2/XTT Assays

Combination assays were performed using varying concentrations of DAPD, DXG, Abacavir and AZT alone or with a fixed concentration of RBV. Five fold serial dilutions of test compound were performed on 96 well plated with the following drug concentrations: DAPD 100 μM, DXG 50 μM, Abacavir 20 μM and AZT 10 μM. The concentrations of RBV used were 1, 5, 10, 20, 40 and 60 μM. Assays were performed in the MT2 cell line as described above in the XXT sensitivity assay section. Addition of 40 and 60 μM RBV, in combination with the compounds listed above, was found to be toxic in these assays, therefore, EC₅₀ values for the compounds were determined in the presence and absence of 1, 5, 10 and 20 μM RBV (Table 2).

TABLE 2
Effects of RBV on the antiviral activity of DAPD, DXG, Abacavir and
AZT in MT2 cells

| | Mean EC₅₀ values (μM) | | | | |
|---|---|---|---|---|---|
| | | 1 μM | 5 μM | | |
| Compound | Control | RBV | RBV | 10 μM RBV | 20 μM RBV |
| DAPD | 18.5 (8)^{a} | 8.2 (2) | 2.9 (2) | 1.6 (4) | 1.3 (4) |
| DXG | 2.65 (8) | 2.05 (2) | 0.58 (2) | 0.5 (2) | 0.22 (2) |
| Abacavir | 4.7 (6) | ND | 6.9 (2) | 6.4 (4) | 5.7 (4) |
| AZT | 1.7 (6) | 2.9 (2) | 4.6 (2) | 5.9 (4) | >10 (4) |

^{a}= number of replicates

Addition of 1, 5, 10 and 20 μM RBV decreased the EC₅₀ values obtained for DAPD and DXG. Table 3 illustrates the fold differences in EC₀ values obtained for each of the compounds in combination RBV.

TABLE 3
Fold differences in EC₅₀ values in combination with RBV in MT2 cells

| Compound | 1 μM RBV | 5 μM RBV | 10 μM RBV | 20 μM RBV |
|---|---|---|---|---|
| DAPD | 2.25 | 6.4 | 11.56 | 14.2 |
| DXG | 1.29 | 4.57 | 5.3 | 12 |
| Abacavir | ND | 0.68 | 0.73 | 0.82 |
| AZT | 0.59 | 0.37 | 0.29 | <0.17 |

Addition of 20 μM RBV had the greatest effect on the antiviral activity of DAPD and DXG with a 14.2 and 12 fold decrease in the apparent EC₅₀ values respectively. Addition of RBV had no effect (less than 2 fold difference in the apparent EC₅₀) on the activity of Abacavir. Addition of 20 μM RBV resulted in a greater than 6-fold increase in the apparent EC₅₀ of AZT indicating that the combination is antagonistic with respect to inhibition of HIV. Similar results were obtained with the addition of 1, 5 and 10, μM RBV, although to a lesser extent than that observed with the higher concentration of RBV.

DAPD Resistant HIV-1 Mutants

The effect of RBV on the activity of DAPD and DXG against mutant strains of HIV was also analyzed (Table 4). The restraint strains analyzed included viruses created by site directed mutagenesis, K65R and L74V, as well as a recombinant virus containing mutations at positions 98S, 116Y, 151M and 215Y. The wild type backbone in which these mutants were created, xxLAI, was also analyzed for comparison. The concentrations of DAPD and DXG tested were as described in the above MT2/XTT combination assay section. RBV was tested in combination with DAPD and DXG at a fixed concentration of 20 μM. The mutant viruses tested all demonstrated increased EC₅₀ values (greater than four fold) for both DAPD and DXG indicating resistance to these compounds. Addition of 20 μM RBV decreased the EC₅₀ values of DAPD and DXG against these viruses. The EC₅₀ values determined for DAPD and DXG in the presence of 20 μM RBV were at least 2.5-fold lower than those obtained for the wild type virus. These results are summarized in Table 4.

TABLE 4
Effects of RBV on the antiviral activity of
DAPD and DXG: Resistant Virus

| | EC₅₀ values (μM) | | | | |
|---|---|---|---|---|---|
| Virus Isolate | DAPD | | DAPD + RBV^{a} | DXG | DXG + RBV |
| K65R | 43.7 | (5.5)^{b} | 0.9 (0.1) | 3.9 (5) | 0.29 (0.4) |
| L74V | 34 | (4) | 0.5 (0.06) | 4.5 (5.6) | 0.25 (0.35) |
| A98S, F116Y, | >100 | (>12) | 2.6 (0.3) | 16 (20) | 0.3 (0.4) |
| Q151M, | | | | | |
| T215Y | | | | | |

^{a}[RBV] = 20 μM
^{b}indicates fold difference from WT

PBMC/p24 Assays

Combination assays were also performed in PBMCs using varying concentrations of DAPD, DXG, Abacavir and AZT alone or with a fixed concentration of RBV. Compound dilutions and assay conditions were as described above. The concentrations of RBV used were 1, 5, 10, 20, 40 and 60 μM. Addition of 40 and 60 μM RBV, in combination with the compounds listed above, was found to be toxic in these assays. The EC₅₀ values determined for the compounds in the presence and absence of 1, 5, 10 and 20 μM RBV are shown in Table 5.

TABLE 5
Effects of RBV on the antiviral activity of DAPD, DXG, Abacavir and
AZT in PMBCs

| | Mean EC₅₀ values (μM) | | | | |
|---|---|---|---|---|---|
| Com- | | 1 μM | 5 μM | 10 μM | 20 μM |
| pound | Control | RBV | RBV | RBV | RBV |
| DAPD | 4.5 (19)^{a} | 2.26 (4) | 0.7 (5) | 0.16 (5) | <0.03 (3) |
| DXG | 0.15 (9) | 0.075 (3) | 0.027 (4) | <0.01 (3) | <0.01 (4) |
| Abacavir | 0.54 (9) | 0.2 (4) | 0.11 (4) | 0.03 (5) | <0.03 (5) |
| AZT | 0.003 (7) | 0.0035 (3) | 0.0026 (3) | 0.0022 (3) | 0.0021 (3) |

^{a}= number of replicates

Addition of 1 μM RBV resulted in a slight decrease (less than 3-fold) in the EC₅₀ of DAPD and DXG and Abacavir, but had no effect on the EC₅₀ value obtained for AZT. These effects became more pronounced with increasing concentrations of RBV. Table 6 illustrates the fold differences in EC₅₀ values obtained for each of the compounds in combination with 1, 5, 10 and 20 μM RBV.

TABLE 6
Fold differences in EC₅₀ values with RBV

| Compound | 1 μM RBV | 5 μM RBV | 10 μM RBV | 20 μM RBV |
|---|---|---|---|---|
| DAPD | 2 | 6.4 | 28 | >150 |
| DXG | 2 | 5.6 | >15 | >15 |
| Abacavir | 2.7 | 4.9 | 18 | >18 |
| AZT | 0.86 | 1.2 | 1.4 | 1.4 |

RBV inhibited the replication of HIV-1 in PBMCs with an EC₅₀ of 20.5 μM. Ribavirin was not toxic to these cells at concentrations up to 1 MM resulting in a therapeutic index of >48. Addition of 20 μM RBV to DAPD, DXG and Abacavir completely inhibited HIV replication in PBMCs at all the concentrations tested but had little effect on the activity of AZT. Addition of lower concentrations of RBV also had a significant effect on the activity of DAPD, DXG and Abacavir. In the MT2 cell line, RBV was not active against HIV replication. Addition of 20 μM RBV decreased the apparent EC₅₀ of DAPD and DXG, 14.2 and 12-fold respectively. Addition of 20 μM RBV had no effect on the activity of Abacavir and resulted in a 6-fold increase in the apparent EC₅₀ of AZT indicating that the combination is antagonistic with respect to inhibition of HIV. Similar results were obtained in MT2s with the addition of 5 and 10 μM RBV, although to a lesser extent than that observed with the higher concentration of RBV. When tested against mutant strains of HIV-1, the combination of 20 μM RBV with DAPD or DXG decreased the EC₅₀ values of these compounds to less than those observed with wild type virus, i.e. the previously resistant virus strains are now sensitive to inhibition by DAPD and DXG. Weislow, O. S., R. Kiser, D. L. Fine, J. Bader, R. H. Shoemaker, and M. R. Boyd. 1989. New soluble formazan assay for HIV-1 cytopathic effects: Application to high-flux screening of synthetic and natural products for AIDS-antiviral activity. J. of NCI. 81:577-586.

VI. Mycophenolic Acid in Combination with DAPD

Mycophenolic acid (MPA) was analyzed in vitro for activity against HIV-1 and for its effects on the in vitro anti-HIV activity of two dGTP analogues, DAPD and DXG. MPA was also evaluated for cytotoxicity in the laboratory adapted cell line MT2 and in peripheral blood mononuclear cells (PBMC). MPA is an inhibitor of the enzyme IMP dehydrogenase. This enzyme is part of the pathway utilized by cells for the de-novo synthesis of GTP. Combination assays were also performed with Abacavir, AZT and FTC.

Cytotoxicity Assays:

MPA was tested for cytotoxicity on the laboratory adapted T-cell line MT2 and in PBMCs using a XTT based assay. The XTT (2,3-bis(2-methoxy-4-nitro-5-sulfophenyl)-5[(phenylamino)carbonyl]-2H-tetrazolium hydroxide) assay is an in vitro colorimetric cyto-protection assay. Reduction of XTT by mitochondria dehydrogenases results in the cleavage of the tetrazolium ring of XTT, yielding orange formazan crystals, which are soluble in aqueous solution. The resultant orange solution is read in a spectrophotometer at a wavelength of 450nM. MPA was prepared in 100% DMSO at a final concentration of 100 mM. For the cytotoxicity assays, a 200 mM solution of MPA was prepared in cell culture media (RPMI supplemented with 10% fetal calf serum, L-Glutamine 1 mg/ml and 20 ug/ml gentamicin) followed by 2 fold serial dilutions on a 96 well plate. Cells were added to the plat at 3×10⁴/well (MTX) and 2×10⁵/well (PBMC) and the plates were incubated for 5 days at 37° C. in a 5% CO₂ incubator (addition of the cells to the plate diluted the compound to a final high concentration of 100 μM). At the end of the 5-day incubation, XTT was added to each well and incubated at 37° C. for 3 hours followed by the addition of acidified isopropanol. The plate was read at 450 nm in a 96 well plate reader. A dose response curve was generated using the absorption values of cells grown in the absence of compound as 100% protection.

MPA was toxic in both cell lines with a 50% cytotoxic does (CC₅₀) of 5.7 μM in the MT2 cell line and 4.5 μM in PBMC. See Table 7.

TABLE 7
Cytotoxicity of MPA

| Cell Type | CC₅₀ |
|---|---|
| MT2 | 5.7 μM |
| PBMC | 4.5 μM |

Sensitivity Assays

XXT Assay

MPA was tested for activity against the xxLAI strain of HIV-1 in the laboratory adapted cell line MT2. Dilutions of MPA were made in cell culture media in a 96 well plate; the highest concentration tested was 1 μM. Triplicate samples of compound were tested. MT2 cells were infected with xxLAI at a multiplicity of infection (MOI) of 0.03 for 3 hours at 37° C. in 5% CO₂. The infected cells were plated at 3.0×10⁴/well into a 96 well plated containing drug dilutions and incubated for 5 days at 37° C. in CO₂. The antiviral activity of MPA was determined using the XTT assay described above. This method has been modified into a susceptibility assay and has been used in a variety of in vitro antiviral tests and is readily adaptable to any system with a lytic virus (Weislow, O. S., et. al. 1989). Using the absorption values of the cell controls as 100% protection and no drug, virus infected cells as 0% protection, a dose response curve is generated by plotting % protection on the Y axis and drug concentration on the X axis. From this curve EC₅₀ values were determined. MPA was not active against HIV-1 in these assays at any of the concentrations tested.

P24 Assay

MPA was also tested for activity against the xxLAI strain of HIV-1 in PBMCs using a p24 based Elisa assay. In this assay, cell supernatants are incubated on microelisa wells coated with antibodies to HIV-1 p24 core antigen. Subsequently, anti-HIV-1 conjugate labeled with horse radish peroxidase is added. The labeled antibody binds to the solid phase antibody/antigen complexes previously formed. Addition of the tetramethylbenzidine substrate results in blue color formation. The color turns yellow when the reaction is stopped. The plates are then analyzed on a plate reader set at 490 nm. The absorbance is a direct measurement of the amount of HIV-1 produced in each well and a decrease in color indicates decreased viral production. Dilutions of MPA were made in cell culture media in a 96 well plate, the highest concentration of MPA tested was 1 μM. PBMC were obtained from HIV-1 negative donors by banding on Ficoll gradients, stimulated with phytohemaglutinin (PHAP) for 48 hours prior to infection with HIV-1, and infected with virus for 4 hours at 37° C. at a MOI of 0.001. Infected cells were seeded into 96 well plates containing 4-fold serial dilutions of MPA. Plates were incubated for 3 days at 37° C. The concentration of virus in each well was determined using the NEN p24 assay. Using the absorption values of the cell controls as 100% protection and drug free, virus infected cells as 0% protection, a dose response curve is generated by plotting % protection on the Y axis and drug concentration on the X axis. From this curve EC₅₀ values were determined.

MPA inhibited HIV-1 replication in PBMCs with a median EC₅₀ of 95 nM±29.

Combination Assays:

The effects of MPA on the in vitro anti-HIV-1 activity of DAPD and DXG were evaluated using the MT2/XTT and PBMC/p24 assays described above. The effects of MPA on the activity of Abacavir, AZT and FTC were also analyzed.

MT2/XTT Assays

Combination assays were performed using varying concentrations of DAPD, DXG, Abacavir, AZT and FTC alone or with a fixed concentration of MPA. Five fold serial dilutions of test compound were performed on 96 well plated with the following drug concentrations: DAPD—100 μM, DXG—50 μM, Abacavir—20 μM and AZT—10 μM, and FTC—10 μM. The concentrations of MPA used were 1, 0.5, 0.25, 0.1, and 0.01 μM. Assays were performed in the MT2 cell line as described in section 3.1. Addition of 1 and 0.5 μμM MPA, in combination with the compounds listed above, was found to be toxic in these assays, therefore, EC₅₀ values for the compounds were determined in the presence and absence of 0.25, 0.1, and 0.01 μM MPA (Table 8).

TABLE 8
Effects of MPA on the antiviral activity of
DAPD, DXG, Abacavir, AZT, and FTC in MT2 cells

| | Mean EC₅₀ values (μM) | | | |
|---|---|---|---|---|
| | | 0.01 μM | | |
| Compound | Control | MPA | 0.1 μM MPA | 0.25 μM MPA |
| DAPD | 20 (5)^{a} | 22 (1) | 4.9 (1) | 1.2 (5) |
| DXG | 2.1 (5) | 2.5 (1) | 0.6 (1) | 0.2 (5) |
| Abacavir | 2.4 (3) | 2.4 (1) | 2.4 (1) | 1.4 (3) |
| AZT | 0.42 (2) | 0.3 (1) | 0.8 (1) | 0.95 (2) |
| FTC | 0.6 (2) | 0.62 (1) | 0.62 (1) | 0.4 (2) |

^{a L = number of replicates}

Addition of 0.01 μM MPA had no effect on the EC₅₀ values obtained for any of the compounds. Table 9 illustrates the fold differences in EC₅₀ values obtained for each of the compounds in combination with 0.1 and 0.25 μM MPA.

TABLE 9
Fold Differences in EC₅₀ Values in Combination with MPA in MT2 cells

| Compound | 0.1 μM MPA | 0.25 μM MPA |
|---|---|---|
| DAPD | 4.1 | 16.7 |
| DXG | 3.5 | 10.5 |
| Abacavir | 1 | 1.7 |
| AZT | 0.5 | 0.44 |
| FTC | 1 | 1.5 |

Addition of 0.25 μM MPA had the greatest effect on the antiviral activity of DAPD and DXG with a 16.7 and 10.5 fold decrease in the apparent EC₅₀ values respectively. Addition of 0.25 μM MPA had little effect on the activity of Abacavir and FTC, less than a 2 fold decrease in the apparent EC₅₀, and resulted in a 2.3 fold increase in the apparent EC₅₀ of AZT indicating that the combination is antagonistic with respect to inhibition of HIV. Similar results were obtained with the addition of 0.1 μM MPA, although to a lesser extent than that observed with the higher concentration of MPA.

DAPD Resistant HIV-1 Mutants

The effect of MPA on the activity of DAPD and DXG against mutant strains of HIV was also analyzed (Table 10). The restraint strains analyzed included viruses created by site directed mutagenesis, K65R and L74V, as well as a recombinant virus containing mutations at positions 98S, 116Y, 151M and 215Y. The wild type backbone in which these mutants were created, xxLAI, was also analyzed for comparison. The concentrations of DAPD and DXG tested were as described in section 4.1. MPA was tested in combination with DAPD and DXG at a fixed concentration of 0.25 μM. DAPD and DXG were active against all of the wild type strains of HIV tested. The mutant viruses tested all demonstrated increased EC₅₀ values for both DAPD and DXG indicating resistance to these compounds. Addition of 0.25 μM MPA decreased the EC₅₀ values of DAPD and DXG against these viruses. These values determined for DAPD and DXG in the presence of 0.25 μM MPA were similar to those obtained for the wild type virus.

TABLE 10
Effects of MPA on the Antiviral Activity of
DAPD and DXG: Resistant Virus

| | EC₅₀ values (μM) | | | |
|---|---|---|---|---|
| Virus Isolate | DAPD | DAPD + MPA^{a} | DXG | DXG + MPA |
| K65R | 41 (6)^{b} | 7.9 (1.1) | 4 (5.6) | 1.2 (1.3) |
| L74V | 39 (4.9) | 6.5 (0.8) | 3.8 (4.2) | 1 (1.1) |
| A98S, F116Y, | 85 (6) | 7 (0.5) | 16 (8.4) | 1.4 (0.7) |
| Q151M, | | | | |
| T215Y | | | | |

^{a}[MPA] = 0.25 μM
^{b}indicates fold difference from WT

PBMC/p24 Assays

Combination assays were also performed in PBMCs using varying concentrations of DAPD, DXG, Abacavir, AZT and FTC alone or with a fixed concentration of MPA. Compound dilutions and assay conditions were as described above. The concentrations of MPA used were 1, 0.5, 0.25, 0.1, and 0.01 μM. Addition of 1 and 0.5 μM MPA, in combination with the compounds listed above, was found to be toxic in these assays. The EC₅₀ values determined for the compounds in the presence and absence of 0.25, 0.1, and 0.01 μM MPA are shown in Table 11.

TABLE 11
Effects of MPA on the antiviral activity of
DAPD, DXG, Abacavir, AZT, and FTC in PMBCs

| | Mean EC₅₀ values (μM) | | | | | | |
|---|---|---|---|---|---|---|---|
| | | | 0.01 μM | | | | 0.25 μM |
| Compound | Control | | MPA | | 0.1 μM MPA | | MPA |
| DAPD | 4.1 | (4)^{a} | 0.9 | (3) | 0.18 | (5) | <0.0002 (2) |
| DXG | 0.14 | (4) | 0.015 | (3) | 0.006 | (5) | <0.0002 (2) |
| Abacavir | 1.2 | (4) | 1.1 | (2) | 0.38 | (3) | <0.0005 (2) |
| AZT | 0.0031 | (3) | 0.0026 | (3) | 0.0021 | (3) | 0.0017 (3) |
| FTC | 0.011 | (3) | 0.008 | (3) | 0.0093 | (3) | 0.006 (2) |

^{a}= number of replicates

Addition of 0.01 um MPA decreased the EC₅₀ for DAPD and DXG but had no effect on the EC₅₀ values obtained for Abacavir, AZT and FTC (less than 2 fold change in EC₅₀). Addition of 0.1 and 0.25 μM MPA decreased the EC₅₀ for DAPD, DXG and Abacavir, but had no effect on the EC₅₀ values obtained for AZT and FTC. Table 12 illustrates the fold differences in EC₅₀ values obtained for each of the compounds in combination with 0.01, 0.1 and 0.25 μM MPA.

TABLE 12
Fold Differences in EC₅₀ Values with MPA

| Compound | 0.01 μM MPA | 0.1 μM MPA | 0.25 μM MPA |
|---|---|---|---|
| DAPD | 4.6 | 22.8 | >50 |
| DXG | 9.3 | 23.3 | >50 |
| Abacavir | 1.1 | 3.2 | >50 |
| AZT | 1.2 | 1.5 | 1.8 |
| FTC | 1.4 | 1.2 | 1.8 |

Mycophenolic acid inhibited the replication of HIV-1 in PBMCs with an EC₅₀ of 0.095 μM. CC₅₀ value obtained for MPA in these cells were 4.5 μM resulting in a therapeutic index of 47. Addition of 0.25 μM MPA to DAPD, DXG and Abacavir completely inhibited HIV replication in PBMCs at all the concentrations tested but had little effect on the activity of AZT and FTC (less than 2-fold change in EC₅₀. Addition of lower concentrations of MPA also had a significant effect on the activity of DAPD, DXG but had little effect on the activity of Abacavir, AZT and FTC. In the MT2 cell line, MPA was not active against HIV replication. Addition of 0.25 μM MPA decreased the apparent EC₅₀ of DAPD and DXG, 16.7 and 10.5-fold respectively. Addition of 0.25 μM MPA had little effect on the activity of Abacavir and FTC and resulted in a 2.3-fold increase in the apparent EC₅₀ of AZT indicating that the combination is antagonistic with respect to inhibition of HIV. Similar results were obtained in MT2s with the addition of 0.1 μM MPA, although to a lesser extent than that observed with the higher concentration of MPA. When tested against mutant strains of HIV-1, the combination of 0.25 μM MPA with DAPD or DXG decreased the EC₅₀ values of these compounds to less than those observed with wild type virus, i.e. the previously resistant virus strains are now sensitive to inhibition by DAPD and DXG.

Concentration of DXG-TP in PBMCs

The effect of mycophenolic acid on the intracellular concentration of DXG-triphosphate (DXG-TP) was evaluated in peripheral blood mononuclear cells (PBMC). PBMC were obtained from HIV negative donors, stimulated with phytohemagluttinin, and incubated at 37° C. in complete media supplemented with various concentrations of DXG (5 μM or 50 μM) in the presence or absence of 0.25 μM mycophenolic acid. PBMC were harvested following 48 or 72 hours of incubation and the intracellular DXG-TP levels determined by LC-MS-MS as described below. Addition of 0.25 μM mycophenolic acid increased the median concentration of intracellular DXG-TP by 1.7-fold as compared to the levels in cells incubated with DXG alone.

The bioanalytical method for the analysis of DXG-TP from peripheral blood mononuclear cells utilizes ion-pair solid phase extraction (SPE) and ion-pair HPLC coupled to electrospray ionization (ESI) mass spectrometry. Pelleted PBMC samples containing approximately 0.5×10⁷ cells are diluted with a solution containing the internal standard (2′, 3′-dideoxycytidine-5′-triphosphate (ddCTP)) and the DXG-TP and ddCTP are selectively extracted using ion-pair SPE on a C-18 cartridge. The DXG-TP and ddCTP are separated with microbore ion-pair HPLC on a Waters Xterra MS C18 analytical column with retention times of about 10 minutes. The compounds of interest are detected in the positive ion mode by ESI-MS/MS on a Micromass Quattro LC triple quadrupole mass spectrometer.

While analyzing DXG-TP PBMC samples, six point, 1/x² weighted, quadratic calibration curves, ranging from 0.008 to 1.65pmoles/10⁶ cells, are used to quantitate samples. Typically, quality control (QC) samples, at two concentrations (0.008 and 1.65pmoles/10⁶ cells), are analyzed in duplicate in each analytical run to monitor the accuracy of the method.

The bioanalytical method has a reproducible extraction efficiency of approximately 80%. The limit of quantitation (LOQ) is 0.008pmoles/10⁶ cells. The range of the assay is 0.008 to 1.65pmoles/10⁶ cells.

This invention has been described with reference to its preferred embodiments. Variations and modifications of the invention, will be obvious to those skilled in the art from the foregoing detailed description of the invention. It is intended that all of these variations and modifications be included within the scope of this invention.

We claim:

1. A pharmaceutical composition for the treatment or prophylaxis of an HIV infection in a host, comprising an effective amount of a β-D-1,3-dioxolanyl purine of the formula: 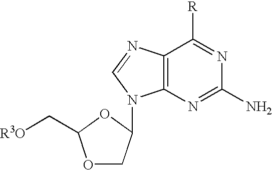
or its pharmaceutically acceptable salt, wherein
R is H, OH, Cl, NH₂ or NR¹R²; R¹ and R² are independently hydrogen, alkyl or cycloalkyl, and R³ is H, alkyl, aryl, acyl, phosphate, including monophosphate, diphosphate or triphosphate or a stabilized phosphate moiety, including a phospholipid, or an etherlipidin combination with at least one inosine monophosphate dehydrogenase (IMPDH) inhibitor, optionally in a pharmaceutically acceptable carrier or diluent.

2. The composition of claim 1, wherein the β-D-1,3-dioxolanyl purine is (−)-(2R,4R)-2-amino-9-[(2-hydroxymethyl)-1,3-dioxolan-4-yl]-adenine (DAPD).

3. The composition of claim 1, wherein the β-D-1,3-dioxolanyl purine is (−)-(2R,4R)-9-[(2-hydroxymethyl)-1,3-dioxolan-4-yl]-guanine (DXG).

4. The composition of any one of claims 1-3, wherein the IMPDH inhibitor is selected from the group consisting of ribavirin, mycophenolic acid, benzamide riboside, tiazofurin, selenazofurin, 5-ethynyl-1-β-D-ribofuranosylimidazole-4-carboxamide (EICAR) and (S)-N-3-[3-(3-methoxy-4-oxazol-5-yl-phenyl)-ureido]-benzyl-carbamic acid tetrahydrofuran-3-yl-ester (VX-497).

5. The composition of claim 4, wherein the IMPDH inhibitors is mycophenolic acid.

6. The composition of claim 4, wherein the IMPDH inhibitors is ribavirin.

7. The composition of claims 1-6, wherein the β-D-1,3-dioxolanyl purine is enantiomerically enriched.

8. The composition of claim 1 in a pharmaceutically acceptable carrier suitable for oral delivery.

9. The composition of claim 1 in a pharmaceutically acceptable carrier suitable for intravenous delivery.

10. The composition of claim 1 in a pharmaceutically acceptable carrier suitable for parenteral delivery.

11. The composition of claim 1 in a pharmaceutically acceptable carrier suitable for topical delivery.

12. The composition of claim 1 in a pharmaceutically acceptable carrier suitable for systemic delivery.

13. A method for the treatment or prophylaxis of a drug resistant strain of HIV infection in a host, comprising administering an effective amount of a β-D-1,3-dioxolanyl purine of the formula: 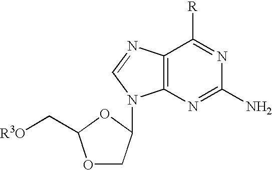
or its pharmaceutically acceptable salt, wherein
R is H, OH, Cl, NH₂ or NR¹R²; R¹ and R² are independently hydrogen, alkyl or cycloalkyl, and R³ is H, alkyl, aryl, acyl, phosphate, including monophosphate, diphosphate or triphosphate or a stabilized phosphate moiety in combination or alternation with an inosine monophosphate dehydrogenase (IMPDH) inhibitors, optionally in a pharmaceutically acceptable carrier or diluent.

14. The method of claim 13, wherein the β-D-1,3-dioxolanyl purine is (−)-(2R,4R)-2-amino-9-[(2-hydroxymethyl)-1,3-dioxolan-4-yl]-adenine (DAPD).

15. The method of claim 13, wherein the β-D-1,3-dioxolanyl purine is (−)-(2R,4R)-9-[(2-hydroxymethyl)-1,3-dioxolan-4-yl]-guanine (DXG).

16. The method of any one of claims 13-15, wherein the IMPDH inhibitor is selected from the group consisting of ribavirin, mycophenolic acid, benzamide riboside, tiazofurin, selenazofurin, 5-ethynyl-1-β-D-ribofuranosylimidazole-4-carboxamide (EICAR) and (S)-N-3-[3-(3-methoxy-4-oxazol-5-yl-phenyl)-ureido]-benzyl-carbamic acid tetrahydrofuran-3-yl-ester (VX-497).

17. The method of claim 16, wherein the IMPDH inhibitor is mycophenolic acid.

18. The method of claim 16, wherein the IMPDH inhibitor is ribavirin.

19. The method of claim 16, wherein the HIV infection is resistant to DAPD and/or DXG.

20. The method of any one of claims 13-19, wherein the host is a human.

21. A method for the treatment or prophylaxis of HIV infection in a host, comprising administering an effective amount of a β-D-1,3-dioxolanyl purine of the formula: 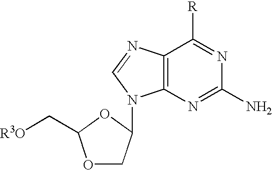
or its pharmaceutically acceptable salt, wherein R is H, OH, Cl, NH₂ or NR¹R²; R¹ and R² are independently hydrogen, alkyl or cycloalkyl, and R³ is H, alkyl, aryl, acyl, phosphate, including monophosphate, diphosphate or triphosphate or a stabilized phosphate moiety in combination or alternation with an inosine monophosphate dehydrogenase (IMPDH) inhibitors, optionally in a pharmaceutically acceptable carrier or diluent.

22. The method of claim 21, wherein the β-D-1,3-dioxolanyl purine is (−)-(2R,4R)-2-amino-9-[(2-hydroxymethyl)-1,3-dioxolan-4-yl]-adenine (DAPD).

23. The method of claim 21, wherein the β-D-1,3-dioxolanyl purine is (−)-(2R,4R)-9-[(2-hydroxymethyl)-1,3-dioxolan-4-yl]-guanine (DXG).

24. The method of any one of claims 21-23, wherein the IMPDH inhibitor is selected from the group consisting of ribavirin, mycophenolic acid, benzamide riboside, tiazofurin, selenazofurin, 5-ethynyl-1-β-D-ribofuranosylimidazole-4-carboxamide (EICAR) and (S)-N-3-[3-(3-methoxy-4-oxazol-5-yl-phenyl)-ureido]-benzyl-carbamic acid tetrahydrofuran-3-yl-ester (VX-497).

25. The method of claim 24, wherein the IMPDH inhibitor is mycophenolic acid.

26. The method of claim 24, wherein the IMPDH inhibitor is ribavirin.

27. The method of any one of claims 21-26, wherein the host is a human.
